(19) United States
(12) Patent Application Publication
(10) Pub. No.: US 2004/0267281 A1
(43) Pub. Date: Dec. 30, 2004

(54) Delivery system for self-expandable diverter

(76) Inventors: Eran Harari, Doar Na Menashe (IL); Avraham Rapaport, Tel Aviv (IL); Oleg Cherniahovsky, Bat Yam (IL); Yuri Davelman, Or Akiva (IL)

Correspondence Address: G.E. EHRLICH (1995) LTD., c/o ANTHONY CASTORINA, SUITE 207, 2001 JEFFERSON DAVIS HIGHWAY, ARLINGON, VA, 22202, US

(21) Appl. No.: 10/603,121

(22) Filed: Jun. 25, 2003

(51) Int. Cl. A61F 11/00

(52) U.S. Cl. 606/108; 623/1.11

(57) ABSTRACT

A delivery system and method for a self-expandable implantable device are provided. The system includes an inner tube, an outer tube and an intermediate tube, wherein the outer tube is designed to be pulled proximally with respect to the intermediate and inner tubes, thereby releasing the implantable device. The system further includes a deployment sleeve with ridges for containment of the diverter, and an elliptical tip for insertion into a blood vessel. The system includes an ellipsoidal tip, which in one embodiment is mechanically connected to the inner tube. The implantable device comprises a diverter stent or graft.

### FIELD AND BACKGROUND OF THE INVENTION

The present invention relates to methods, systems and devices for in vivo delivery of a diverter and, more particularly, to methods, systems and devices for delivery of a self-expandable diverter.

A diverter is a prosthetic device for placement within a body lumen, usually a blood vessel, for diverting blood clots from one branch to another. More particularly, a diverter is designed to divert blood clots away from the critical internal carotid artery leading to the brain, thereby preventing stroke. This is accomplished by deflecting the flow of embolic material into the external carotid artery instead. An example of a diverter is disclosed in U.S. Pat. No. 6,348,063 to Yassour et al, incorporated herein by reference in its entirety.

A diverter is inserted into the body in a compressed form, and allowed to expand once it reaches the site of deployment, similar to a stent. Conventional stent systems employ a balloon catheter on which the stent is mounted. Expansion of the balloon causes the stent to expand. Once the stent is fully expanded, the catheter system is removed from the body, leaving the stent in place.

Examples of conventional balloon stents in the patent literature include several designs, generally featuring a straight, tubular configuration, with a cellular structure patterned throughout. For example, a stent disclosed by Palmaz, in U.S. Pat. No. 5,102,417, has a series of elongated tubular members having a plurality of slots, wherein the tubular members are connected by a flexible connecting member. In a stent disclosed by Israel, in U.S. Pat. No. 5,733,303, the stent is described as a slotted tube having a pattern shape with first and second patterns, wherein the second pattern runs perpendicular to the first pattern. In each of these examples, the stent is disposed on a balloon catheter for delivery to a site within the lumen.

Another variety of stents is the self-expandable type, in which a balloon is not used to deploy the stent. U.S. Pat. No. 4,732,152 to Wallsten et al. discloses a device for implantation of an expandable self- fixing prosthesis. The device has a prosthesis in a radially contracted state, and means for maintaining and releasing the prosthesis. The means for maintaining and releasing the prosthesis includes a flexible hose folded inside itself to form a double-walled section radially surrounding the prosthesis. Axial relative movement of the ends of the hose allows the stent to be released.

In U.S. Pat. No. 5,201,757 to Heyn et al., there is disclosed an apparatus for deploying a radially self-expanding stent starting from the medial portion of the stent. The apparatus includes proximal and distal sleeves containing proximal and distal end portions of the stent in a reduced radius delivery configuration. The sleeves are moved axially with respect to one another to permit radial self-expansion of the stent only over its medial region, while the sleeves continue to contain the axially outward regions of the stent. Eventually, the stent becomes fully free of the sleeves resulting in radial expansion over the entire stent length.

U.S. Pat. No. 5,571,168 to Toro discloses a delivery system, which has three concentric shafts: an inner shaft for carrying a medical device, a middle pull back shaft and an outer stiffening shaft. The inner and outer shafts are connected together at the proximal end of the delivery system to preclude the inner shaft from moving axially relative to the outer shaft as the middle pull back shaft is retracted, allowing for accurate placement of the medical device.

While all of the above references disclose delivery systems for expandable stents, a diverter differs from a stent in several respects. Since stents are designed to support artery walls in an open state, the slots are built in a way to most effectively provide support, maximizing stiffness. In addition, stents are designed to provide radial forces that are strong enough to hold up a vessel and hold back any obstructions within the vessel. A diverter, on the other hand, is a mesh configuration designed to minimize radial forces so as to minimize damage to an artery wall. Accordingly, more wires are used, wires are thinner (15 μm˜60 μm as compared to 90 μm˜150 μm for stents), braid angles are larger, and window size is smaller. Most significantly, a diverter is more delicate in structure than a stent and as such, must be delivered with greater care. Thus, each one of the cited prior art patents fail to teach a self-expandable diverter delivery system which would result in little or no damage to the diverter upon its release, as well as to the body lumen in which it is released.

There is thus a great need for and it would be highly advantageous to have a diverter delivery system that would minimize damage to the diverter upon its release as well as to the body lumen upon its entry and release.

### SUMMARY OF THE INVENTION

According to one aspect of the present invention there is provided a system for delivery of an implantable device. The system includes an inner tube on which the implantable device is mounted, an outer tube enclosing the inner tube and the implantable device, and an intermediate tube mounted between the inner tube and the outer tube, the intermediate tube including a material to ease sliding of the outer tube.

In a preferred embodiment the implantable device is a diverter.

According to further aspects of the present invention, there is provided a deployment sleeve for a self-expandable implantable device, wherein the deployment sleeve is generally cylindrical and hollow so as to be positioned on a tube, and having ridges, wherein each ridge is formed along a longitudinal axis of the sleeve.

According to yet further aspects of the present invention, there is provided a device for introducing into a body lumen. The device includes an inner tube having a proximal end and a distal end, an outer tube enclosing the inner tube, an intermediate tube mounted between the inner tube and the outer tube, wherein the distal end of the intermediate tube is located proximal to the distal end of the inner tube, and a stopper attached to the inner tube at the distal end of the intermediate tube.

According to yet further aspects of the present invention, there is provided a device for introducing into a body lumen. The device includes an elongated member having a proximal end and a distal end, and a tip having an ellipsoidal shape, wherein the tip is located at the distal end of the elongated member. In a preferred embodiment, the tip is attached to the distal end of the elongated member mechanically. The mechanical attachment includes a band located on the elongated portion of the tip, wherein the band has an outer diameter which is less than a diameter of the widest portion of the ellipsoidal shape, and a flared portion on the distal end of the elongated member, wherein the ellipsoidal shape is situated over the flared portion.

According to yet further aspects of the present invention, there is provided a method for positioning an implantable device within a body lumen. The method includes providing a diverter delivery system having an inner tube, an intermediate tube enclosing at least a portion of the inner tube and attached to the inner tube, and an outer tube enclosing the intermediate tube and the inner tube, and a diverter between the inner and outer tubes, inserting the delivery system into the body lumen, and pulling the outer tube proximally with respect to the inner tube, the pulling being eased by the relatively low friction intermediate tube so as to release the diverter.

According to still another aspect of the present invention, there is provided a method for preventing entanglement of an implantable device within a delivery system. The method includes providing a delivery system having an inner tube having a diverter positioned on the inner tube and a stopper positioned on the inner tube proximal to the diverter, having an outer tube positioned over the inner tube, and further having an intermediate tube attached to the inner tube, wherein a distal end of the intermediate tube at least partially covers the stopper, and sliding the intermediate tube distally with respect to the outer tube so that the distal end of the second tube flares outward over the stopper, thereby coming in contact with the outer tube and forming a divider between the implantable device and a proximal portion of the delivery system.

According to yet another aspect of the present invention, there is provided a method for positioning an implantable device within a delivery system. The method includes providing a sleeve with indentations on a distal end of an inner tube, placing the implantable device at least partially on the sleeve wherein part of the implantable device sits within the indentations, and providing an outer tube to enclose the implantable device and the inner tube.

The present invention successfully addresses the shortcomings of the presently known configurations by providing a system and method for delivering a self-expandable implantable device which is designed to avoid damage to the implantable device, as well as the blood vessel.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic view of a system in accordance with one embodiment of the present invention;

FIG. 2 is a cross sectional illustration of the system of FIG. 1;

FIG. 3 is an expanded view of an intermediate tube with respect to inner and outer tubes within the system of FIGS. 1 and 2, depicting the distal end of the intermediate tube positioned over a stopper;

FIGS. 4a-4c are schematic illustrations of a deployment sleeve, in accordance with one embodiment of the present invention;

FIGS. 5a-5c are schematic illustrations of a self-expandable implantable device contained within an outer tube and progressing through the various stages of release;

FIG. 6 is an illustration of an ellipsoidal tip; and

FIG. 7 is a cross sectional illustration of the tip of FIG. 6.

### DESCRIPTION OF THE PREFERRED EMBODIMENTS

The present invention is of a device and method for deploying a self-expandable implantable device such as a diverter.

Specifically, the present invention can be used to deliver an implantable device such as a diverter and allow it to deploy within a blood vessel without causing damage to the device or to the blood vessel. A diverter is designed to be implanted within a blood vessel, such as an artery, and specifically can be used to divert a blood clot away from a critical blood vessel, such as the internal carotid artery. It should be readily apparent that while the description and advantages are presented with respect to a diverter, a similar construction and operation is possible for the use of a delivery device for a stent, graft or any other implantable device as well.

The principles and operation of a diverter delivery system according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

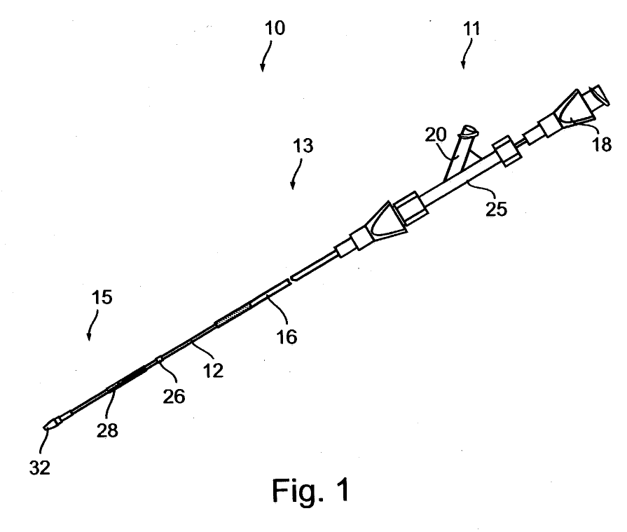

Turning now to the drawings, FIG. 1 illustrates a delivery system 10 in accordance with one embodiment of the present invention. For the purposes of the present application, the proximal end is defined as the end that is outside of the patient's body (closest to the practitioner), shown in FIG. 1 on the right-hand side, while the distal end is the portion that enters the body first, shown in FIG. 1 on the left-hand side. Delivery system 10 includes a proximal hub 11, a middle section 13 and a distal loading area 15. In FIG. 1, middle section 13 is depicted with a break in the middle, since middle section 13 is proportionately very long, comprising most of the length of delivery system 10, and as such is not depicted in its entirety. In one embodiment, proximal hub 11 is between 150 and 400 mm in length, middle section 13 is 900 to 1500 mm in length, and distal loading area 15 is 50 to 200 mm in length. In a preferred embodiment, proximal hub 11 is approximately 200-250 mm in length, middle section 13 is approximately 1100-1200 mm in length, and distal loading area 15 is approximately 100-150 mm in length. It should be readily apparent that each of these sections may be any suitable length, and that the measurements disclosed above are for exemplary purposes only.

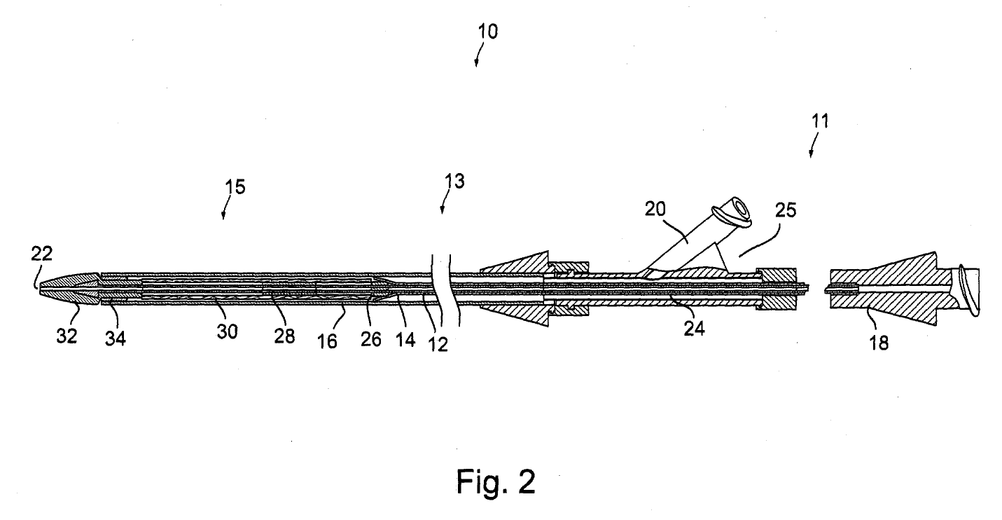

Proximal hub 11 further comprises a stiffening tube 24, preferably comprised of a metal, for support of a proximal portion of an inner tube 12, a hemostatic Y valve 25 for regulating blood leakage, an inner tube port 18 and an outer tube port 20. The Y valve may be, for example, any standard Y valve, such as one from Qosina (Edgewood, N.Y., USA, catalog number 88416). Middle section 13 comprises tubing, as will be discussed in greater detail further hereinbelow. Distal loading area 15 comprises tubing, a sleeve placed thereon 28, and a tip 32. Distal loading area 15 further optionally comprises a stopper 26, proximal to sleeve 28. In a final configuration, an implantable device (not shown) such as a diverter sits on sleeve 28, and intermediate and outer tubes are present as well, as will be described in further detail with reference to FIG. 2.

Reference is now made to FIG. 2, which is a cross-section illustration of delivery system 10 depicted in FIG. 1, and further including a diverter 30 and intermediate and outer tubes 14, 16. As shown in FIG. 2, in a preferred embodiment diverter 30 is partially situated on sleeve 28, but extends proximally and distally past sleeve 28 so that only a middle portion of diverter 30 is directly in contact with sleeve 28. Beginning at a proximal end of diverter 30, an intermediate tube 14 is situated on top of inner tube 12, optionally having a stopper 26 at its distal end to separate between intermediate tube 14 and diverter 30. An outer tube 16 is situated on top of at least part of system 10, sandwiching diverter 30, sleeve 28, intermediate tube 14, and optionally stopper 26 between outer tube 16 and inner tube 12. As indicated above the term diverter is meant to be illustrative only, and is not meant to be limiting in any way. The invention may be practiced with any implantable device including stents or grafts.

A particular feature of the present invention is that by using a combination of inner, intermediate and outer tubes 12, 14, 16, as well as sleeve 28, which holds diverter 30 in place, it is not necessary to push or pull diverter 30, thereby potentially causing damage to the fragile device. Rather, outer tube 16 is gently pulled back over a low friction surface (intermediate tube 14), while diverter 30 remains in place over a relatively high friction surface (sleeve 28). As outer tube 16 is pulled back, diverter 30 automatically deploys, without any unnecessary or unwanted tugging.

As shown in FIG. 2, inner tube 12 has an inner diameter that forms a guidewire lumen 22 for passage of a standard guidewire there through. In a preferred embodiment, the dimension of the inner diameter of inner tube 12 is 0.5-2 mm, and in an exemplary preferred embodiment is approximately 1 mm. Inner tube 12 extends the entire length of system 10, and is designed to facilitate smooth guidewire insertion and movement. The proximal end of inner tube 12 ends in proximal hub 11 proximal to inner tube port 18. The distal end of inner tube 12 has a tip 32, secured by band 34, which will be described in greater detail further hereinbelow. Inner tube 12 may be made of a variety of materials. In one embodiment, it comprises a polymer, such as PEEK or polyimide, for example. In another embodiment, inner tube 12 comprises a metal, such as Nitinol or thin-walled super alloys, for example. In yet another embodiment, inner tube 12 is made from a composite of materials, in concentric layers or with reinforcement braided wires, for example.

To compensate for the flexibility of inner tube 12 at the proximal end of system 10, a stiffening tube 24 covers inner tube 12. In a preferred embodiment, stiffening tube 24 extends between outer tube port 20 or Y valve 25 and inner tube port 18, to prevent collapse of the exposed proximal portion of inner tube 12. Stiffening tube 24 is preferably comprised of a metal, for example stainless steel, titanium alloy, or others.

Intermediate tube 14 serves to ease sliding between inner and outer tubes 12 and 16. Intermediate tube 14 is held in place on inner tube 12. In a preferred embodiment, intermediate tube 14 extends from a stopper 26 to stiffening tube 24. In an alternative embodiment (not shown), intermediate tube 14 extends from stopper 26 to inner tube port 18, extending over stiffening tube 24. Stopper 26 may or may not be included. In a preferred embodiment, intermediate tube 14 comprises PTFE or a similar material to reduce friction.

Intermediate tube 14 serves as a low friction device and runs throughout the tubing system. The use of intermediate tube 14 obviates the need for a friction-reducing liner on the outer tube, as is found in many other prior art devices. It should be noted, however, that a liner may optionally be used with the device of the present invention as well. A further advantage in using intermediate tube 14 is that a single tube length can be used for various diverter sizes and lengths, since changing the location of stopper 26 and thus the end location of intermediate tube 14 within the system can compensate for changes in diverter size or length. A further advantage in using intermediate tube 14 is that it reduces the gap between inner and outer tubes 12 and 16, thus making the shaft less prone to kinking when it is bent. At the same time, since the length of intermediate tube 14 ends just proximal to distal loading area 15, enough of a gap is still present so as to accommodate diverter 30. Furthermore, markers may be placed on the distal end of intermediate tube 14, allowing for better visualization of the system.

Outer tube 16 extends from tip 32 to proximal hub 11, and is slidably positionable with respect to inner tube 12. Its purpose is to contain the self-expandable diverter in its unexpanded state on inner tube 12, enabling it to remain contained until the desired location is reached and only then allowing its release and consequent expansion. In a preferred embodiment, outer tube 16 comprises a biocompatible polymer, such as polyamide, PTFE, polyether block amide (Pebax), Polyimide, PEEK, or any other suitable polymer, and may further comprise reinforcement stainless steel braided wires. Furthermore, in alternative embodiments, outer tube 16 further comprises an inner layer of PTFE to reduce friction. Outer tube 16 ends with a standard luer lock. The distal end of outer tube 16 is transparent to facilitate inspection of diverter 30 at the factory or prior to use. It may also optionally have a radiopaque marker band at its distal end to facilitate visualization of the deployment procedure.

Another feature according to a preferred embodiment of the present invention is that outer tube 16 does not need to be transparent along the entire length of delivery system 10, except for the most distal portion: distal loading area 15. Other prior art systems frequently use either a transparent tube, in which there is no braided reinforcement of the shaft, or alternatively, a reinforced shaft where the diverter is not visible. In the present invention, the shaft is reinforced where strength is needed, and is transparent where strength is less important. This feature allows for quality control post-manufacturing and final inspection before the system is used in the body of a person, while permitting more freedom in the selection of the non-transparent section materials.

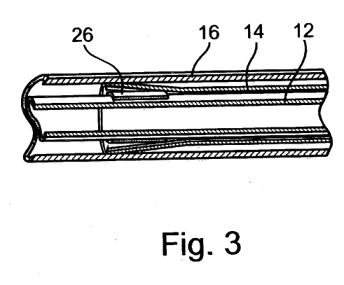

Reference is now made to FIG. 3, which depicts the distal end of intermediate tube 14 positioned over stopper 26. Stopper 26 serves as a border between intermediate tube 14 and a self-expandable diverter (not shown), which is situated slightly distal to stopper 26. Stopper 26 allows intermediate tube 14 to widen at its distal end so that diverter (not shown) does not become entangled or caught between inner tube 12 and outer tube 16, essentially acting as a washer. The widened end of intermediate tube 14 further accommodates the tolerance of tube production, with the widened end adjusting to match the actual inner diameter of outer tube 16. In addition, in one embodiment of the present invention, stopper 26 is made from radiopaque material, allowing it to be viewed fluoroscopically under X-ray during a procedure. In this way, the proximal end of diverter 30 can be visualized, which aids in proper positioning of diverter 30 within the body lumen.

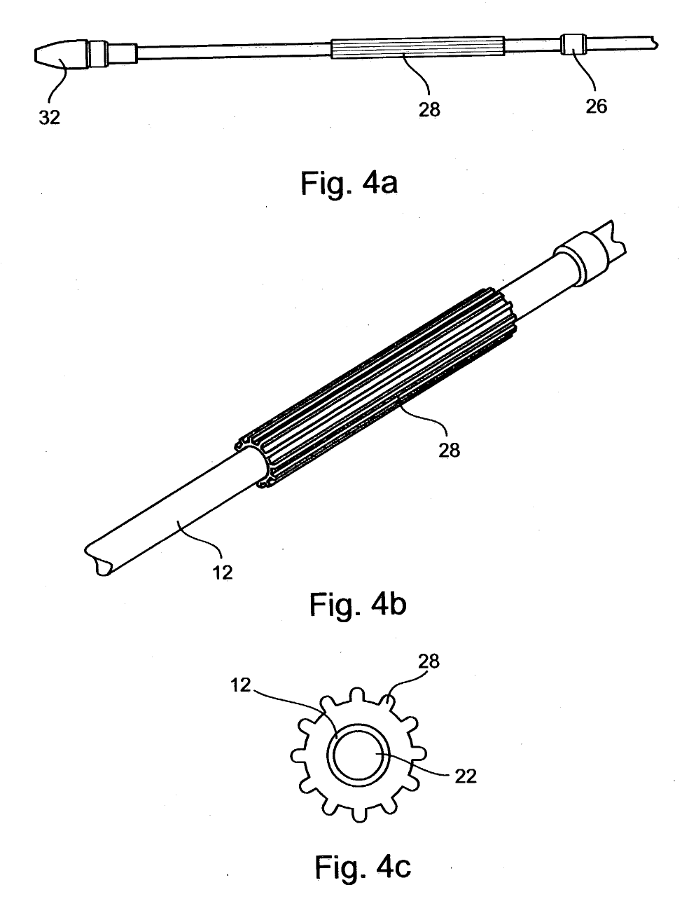

Reference is now made to FIGS. 4a-4c, which are schematic illustrations of a deployment sleeve 28, in accordance with a further embodiment of the present invention. Deployment sleeve 28 is positioned on top of inner tube 12. In one embodiment, deployment sleeve 28 is 5-20 mm in length. In a preferred embodiment, deployment sleeve 28 is 10-16 mm in length. In an exemplary embodiment, deployment sleeve 28 comprises elastomeric material. The elastomeric material presents a pliant surface to diverter 30, allowing diverter 30 to lie within the surface thereof. As can be seen in FIG. 4b and in cross section in FIG. 4c, deployment sleeve 28 comprises ridges or indentations around its circumference. This configuration provides a secure site for positioning diverter 30 and providing friction to keep diverter 30 in place. In alternative embodiments, sleeve 28 comprises diamond shape or any other shaped indentations.

In a preferred embodiment, deployment sleeve 28 is shorter in length than diverter 30. In accordance with an embodiment of the present invention, deployment sleeve 28 is 5-60 mm, while diverter 30 is 50-90 mm. In accordance with an exemplary embodiment, deployment sleeve 28 is 10-16 mm and diverter 30 is 70-90 mm in length. Thus, diverter 30 only partially sits on and within deployment sleeve 28. In this way, more room is available for diverter 30 in the adjacent hollow sections between inner and outer tubes 12 and 16, and there is less friction between diverter 30 and outer tube 14, thereby reducing the deployment force. The friction between diverter 30 and outer tube 14 is primarily a function of the length of deployment sleeve 28, as preferably the outer diameter of diverter 30, which is seated on deployment sleeve 28, is nominally the same as the inner diameter of outer tube 14. Thus, the distal end of system 10 may comprise a relatively small diameter while still allowing room for the diverter, thereby facilitating insertion of system 10 within a small body lumen. In an exemplary embodiment diverter 30 is comprised of braided fine wire, and thus the gap between deployment sleeve 28 and outer tube 14 is nominally twice the wire diameter.

In one embodiment of the present invention, deployment sleeve 28 comprises radiopaque material. In this way, a user can visualize the exact location of diverter 30 without having to include a separate radiopaque marker on inner tube 12 or outer tube 16.

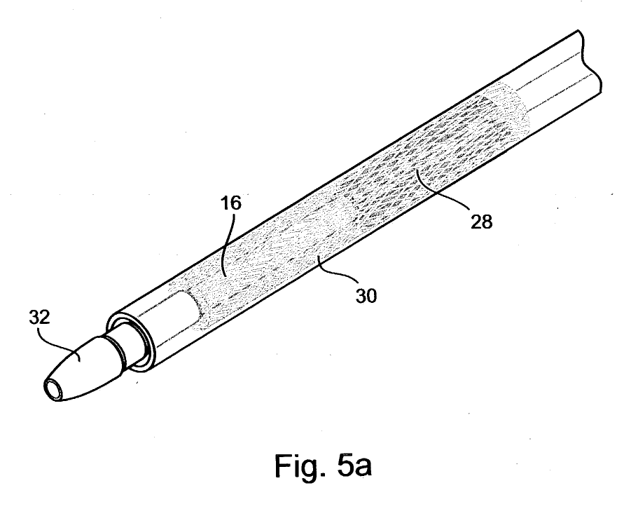

Reference is now made to FIGS. 5a-5c, which show deployment sleeve 28 with diverter 30 in various states of being released from between deployment sleeve 28 and outer tube 16. In FIG. 5a, diverter 30 is shown situated between deployment sleeve 28 and outer tube 16. Inner tube 12 and tip 32 are shown for reference. In FIG. 5b, diverter 30 is shown partially released, caused by outer tube 16 being moved proximally relative to deployment sleeve 28 thus exposing diverter 30. Once diverter 30 is free of outer tube 16, it expands radially as shown in FIG. 5c until it contacts the inner walls (not shown) of the body lumen in which it is placed.

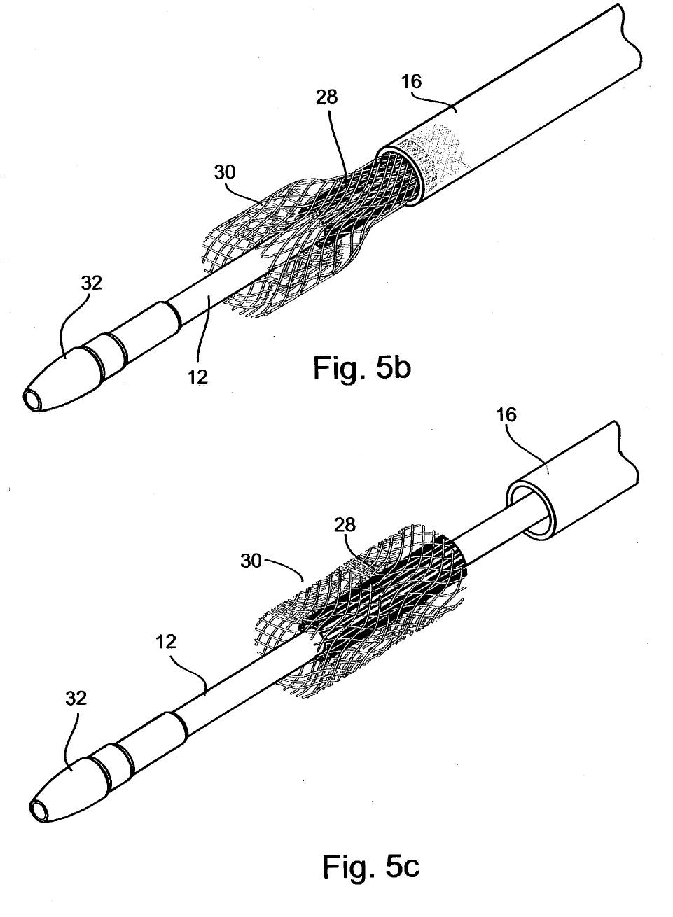

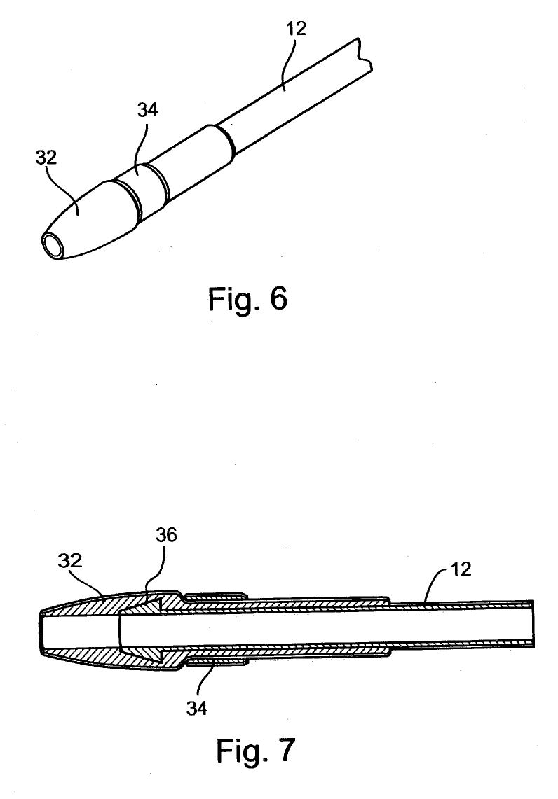

Reference is now made to FIGS. 6 and 7, which are schematic and cross-section illustrations of tip 32, in accordance with an embodiment of the present invention. The shape and configuration of tip 32 eliminate many problems commonly associated with tips attached to a system and entering a body. Specifically, the shape is ellipsoidal, rather than conical as is the case in tips found in the prior art. Use of an ellipsoidal shape in place of the conical shape found in the prior art increases the amount of material thus strengthening the tip, while still utilizing the same advantageous material known to the art. Use of an ellipsoidal shape further maintains the critical dimension of the distal and proximal ends of the tip as known in the prior art with a resultant increase in tip strength and stiffness. In addition, the tip is attached by mechanical means rather than by glue, as in the prior art, mitigating the possibilities of the tip dislodging from within the blood vessel.

Specifically, as can be seen in FIG. 7, the distal end of inner tube 12 exhibits outward flare 36. Tip 32 is positioned on top of the flare 36 so as to prevent movement of tip 32 with respect to inner tube 12. In addition, the proximal end of tip 32 lies straight on top of the inner tube. In a preferred embodiment band 34 is placed on this straight portion to prevent movement of tip 32 with respect to the inner tube 12. In an additional embodiment, band 34 is radiopaque so as to provide a view of the distal end of the system to a user.

The basic operating principle of the system is as follows: a guidewire is inserted into a body lumen, which, in a preferred embodiment, is a blood vessel. A delivery system is then advanced over the guidewire so as to be positioned within the body lumen at a desired location. When in position, outer tube 16 is pulled proximally with respect to intermediate tube 14, which is secured to inner tube 12; diverter 30 is exposed and automatically expands so as to cover the desired section of the body lumen. This system and method differ from prior art systems and methods since in the present invention, there is no contact between the proximal end of the diverter and the releasing mechanism, thus eliminating possible damage to the diverter strands. Furthermore, rather than risking damage to the diverter by pushing it or pulling it, the diverter remains in place due to friction on the sleeve, and is released by pulling of the outer tube, rather than by any action on the diverter itself. In one embodiment the flared portion of intermediate tube 14 only pushes the proximal undeployed section of diverter 30 after the proximal end of deployment sleeve 28 has cleared outer tube 16, thus requiring minimal force.

It is appreciated that certain features of the embodiment may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for delivery of an implantable device, the system comprising:
an inner tube on which the implantable device is mounted;
an outer tube enclosing said inner tube and said implantable device; and
an intermediate tube mounted between said inner tube and said outer tube, said intermediate tube comprised of a material to ease sliding of said outer tube.

2. The system of claim 1, wherein said implantable device is selected from the group consisting of a diverter, a stent and a graft.

3. The system of claim 1, further comprising a deployment sleeve located on said inner tube for holding said implantable device in place prior to sliding of said outer tube.

4. The system of claim 3, wherein said deployment sleeve comprises indentations.

5. The system of claim 4, wherein said indentations are parallel ridges.

6. The system of claim 3, wherein said deployment sleeve comprises elastomeric material.

7. The system of claim 1, wherein said implantable device is self-expandable.

8. The system of claim 7, wherein said self-expandable implantable device is braided.

9. The system of claim 7, wherein said self-expandable implantable device has a shape memory.

10. The system of claim 7, wherein said self-expandable implantable device is released for expansion by pulling said outer tube proximally with respect to said intermediate tube.

11. The system of claim 1, wherein said intermediate tube is securably connected to said inner tube.

12. The system of claim 1, wherein said intermediate tube further comprises radiopaque markers.

13. The system of claim 1, wherein said intermediate tube is outwardly flared at its distal end.

14. The system of claim 13, further comprising a stopper at said outwardly flared distal end of said intermediate tube, said stopper being secured to said inner tube.

15. The system of claim 14, wherein said stopper is radiopaque.

16. The system of claim 1, further comprising an ellipsoidal tip.

17. The system of claim 16, wherein said tip further comprises an elongated portion and is mechanically attachable to said inner tube.

18. The system of claim 17, wherein said mechanical attachment comprises:
a band located on said elongated portion, wherein said band has an outer diameter which is less than a diameter of the widest portion of said ellipsoidal tip; and
a flared portion on said distal end of said hollow inner tube, wherein said ellipsoidal tip is situated over said flared portion.

19. The system of claim 18, wherein said band is radiopaque.

20. The system of claim 18, wherein said flared portion and said hollow inner tube are fabricated from one piece.

21. The system of claim 1, wherein said outer tube is at least partially transparent.

22. The system of claim 21, wherein said outer tube is transparent along its length from its proximal end until a proximal end of said diverter.

23. The system of claim 1, wherein said inner tube is hollow.

24. The system of claim 23, wherein said inner tube has an inner diameter of 0.1 to 1 mm.

25. A deployment sleeve for a self-expandable device, wherein said deployment sleeve is generally cylindrical and hollow so as to be positioned on a tube, and having ridges, wherein each ridge is formed along a longitudinal axis of said sleeve.

26. The sleeve of claim 25, wherein said ridges are parallel to one another.

27. The sleeve of claim 25, wherein said deployment sleeve comprises elastomeric material.

28. The sleeve of claim 25, wherein said tube is a hollow inner tube, said tube and said deployment sleeve being positionable within a body lumen.

29. The sleeve of claim 25, wherein said body lumen is a blood vessel.

30. The sleeve of claim 28, wherein said hollow inner tube has an inner diameter of 0.1-1 mm.

31. The sleeve of claim 28, wherein said inner tube further comprises an ellipsoidal tip at its distal end.

32. The sleeve of claim 31, wherein said tip is mechanically attached to said inner tube.

33. The sleeve of claim 25, further comprising a self-expandable implantable device positioned on said deployment sleeve wherein a portion of said implantable device lies within said ridges.

34. The sleeve of claim 33, further comprising an outer tube surrounding said implantable device, wherein displacement of said outer tube with respect to said implantable device allows said implantable device to be expanded.

35. The sleeve of claim 25, wherein said sleeve is positioned near a distal end of an implantable device delivery system.

36. The sleeve of claim 33, wherein said sleeve is shorter in length than said implantable device and wherein only a portion of said implantable device is positioned on said sleeve.

37. A device for introducing into a body lumen, said device comprising:
an inner tube having a proximal end and a distal end;
an outer tube enclosing said inner tube;
an intermediate tube mounted between said inner tube and said outer tube, said intermediate tube having a proximal end and a distal end, wherein said distal end of said intermediate tube is located proximal to said distal end of said inner tube; and
a stopper attached to said inner tube at said distal end of said intermediate tube.

38. The device of claim 37, wherein said distal end of said intermediate tube is flared outwardly over said stopper.

39. The device of claim 37, wherein said body lumen is a blood vessel.

40. The device of claim 37, wherein said inner tube is a hollow tube and wherein an inner diameter of said hollow tube is 0.1-1 mm.

41. The device of claim 37, wherein said intermediate tube comprises PTFE.

42. The device of claim 37, wherein said stopper is radiopaque.

43. The device of claim 37, further comprising a self-expandable implantable device positioned between said inner and outer tubes in a location distal to said stopper.

44. The device of claim 43, wherein said implantable device is positioned on a sleeve attached to said inner tube.

45. The device of claim 44, wherein said sleeve comprises elastomeric material.

46. The device of claim 44, wherein said sleeve is shorter in length than said implantable device.

47. The device of claim 43, wherein said self-expandable implantable device is braided.

48. The device of claim 37, wherein said inner tube further comprises an ellipsoidal tip.

49. The device of claim 48, wherein said ellipsoidal tip is attached to said inner tube mechanically.

50. A device for introducing into a body lumen, said device comprising:
an elongated member having a proximal end and a distal end; and
a tip having an ellipsoidal shape, wherein said tip is located at said distal end.

51. The device of claim 50, wherein said tip has an elongated portion proximal to said ellipsoidal shape, and wherein said tip is attached to said distal end of said elongated member mechanically.

52. The device of claim 51, wherein said mechanical attachment comprises:
a band located on said elongated portion of said tip, wherein said band has an outer diameter which is less than a diameter of the widest portion of said ellipsoidal shape; and
a flared portion on said distal end of said elongated member, wherein said ellipsoidal shape is situated over said flared portion.

53. The device of claim 52, wherein said band is radiopaque.

54. The device of claim 50, wherein said body lumen is a blood vessel.

55. The device of claim 50, wherein said elongated member is part of an implantable device delivery system.

56. The device of claim 55, wherein said elongated member is a hollow tube, and wherein an inner diameter of said hollow tube is 0.1-1 mm.

57. The device of claim 55, wherein said elongated member further comprises a self-expandable implantable device.

58. A method for positioning an implantable device within a body lumen, the method comprising:
providing a delivery system having an inner tube, an intermediate tube at least partially covering said inner tube, an outer tube enclosing said inner tube and said intermediate tube, and an implantable device between said inner and outer tubes;
inserting said delivery system into the body lumen; and
pulling said outer tube proximally with respect to said inner tube so as to release the implantable device.

59. The method of claim 58, wherein said implantable device is a diverter, stent or graft.

60. The method of claim 58, wherein said body lumen is a blood vessel.

61. The method of claim 58, wherein said pulling said outer tube is eased by the relatively low friction of said intermediate tube.

62. The method of claim 61, further comprising a stopper at a distal end of said intermediate tube, wherein the distal end of the intermediate tube at least partially encloses said stopper.

63. The method of claim 59, further comprising allowing the implantable device to self-expand.

64. The method of claim 63, wherein said implantable device comprises braided components, and wherein said self-expansion occurs due to extension of said braided components upon removal of said outer tube.

65. A method for preventing entanglement of an implantable devicewithin a delivery system, the method comprising:
providing a delivery system having an inner tube having a diverter positioned on the inner tube and a stopper positioned on the inner tube proximal to said implantable device, having an outer tube positioned over the inner tube, and further having an intermediate tube attached to the inner tube, wherein a distal end of the intermediate tube at least partially covers the stopper; and
sliding the intermediate tube distally with respect to said outer tube so that the distal end of the second tube flares outward over the stopper, thereby coming in contact with the outer tube and forming a divider between the implantable device and a proximal portion of the delivery system.

66. The method of claim 65, further comprising visualizing said system within a body by providing a radiopaque material for said stopper.

67. The method of claim 65, further comprising easing said sliding by providing a low friction material for said intermediate tube.

67. The method of claim 64, wherein said sliding is done from a proximal end of said delivery system.

68. A method for positioning an implantable device within a delivery system, the method comprising:
providing a sleeve with indentations on a distal end of an inner tube;
placing the implantable device at least partially on the sleeve wherein part of the implantable device sits within the indentations; and
providing an outer tube to enclose the implantable device and the inner tube.

69. The method of claim 68, wherein said implantable device is a diverter, stent or graft.

70. The method of claim 68, wherein said indentations are ridges in a longitudinal direction.

71. The method of claim 68, wherein said implantable device is longer than said sleeve.
